(19) United States
(12) Patent Application Publication
(10) Pub. No.: US 2014/0034068 A1
(43) Pub. Date: Feb. 6, 2014

(54) CONFIGURABLE ORTHOPEDIC DEVICE

(71) Applicants: Thomas J. Picar, Sunland, CA (US); Godofredo C. Gatus, II, Tarzana, CA (US); Hoang K. Nguyen, Salisbury, NC (US)

(72) Inventors: Thomas J. Picar, Sunland, CA (US); Godofredo C. Gatus, II, Tarzana, CA (US); Hoang K. Nguyen, Salisbury, NC (US)

(21) Appl. No.: 13/568,107

(22) Filed: Aug. 6, 2012

(51) Int. Cl. A61F 13/06

(52) U.S. Cl. 128/888

(57) ABSTRACT

An orthopedic walking boot and method of use for off-loading plantar ulcers is disclosed. The orthopedic walking boot may be particularly effective for off-loading the plantar ulcers of diabetic patients with impeded mobility due to age or illness who cannot be treated with a total contact cast. Embodiments of the orthopedic walking boot presented offer a configurable insole of removable cells seated in a rigid support frame. The cell array may be custom fitted to the size, shape, and location of the patient's planter ulcer.

### FIELD

This invention relates generally to an orthopedic device, and more specifically to an orthopedic walking boot to facilitate healing of diabetic foot ulcers. The orthopedic boot may have a configurable cell array for offloading a diabetic foot. The invention may also have applications relevant to musculoskeletal disorders of the foot and ankle. This walking boot will expedite the healing time of the foot ulcer while stabilizers in the structure provides support, allowing the patient to remain mobile during the healing process.

### BACKGROUND

A simple sugar, glucose, is produced when food is broken down for energy during digestion. Diabetes is a chronic disease in which high levels of glucose is present in the blood. Insulin, a hormone produced in the pancreas, signals muscle, fat and liver cells to take up glucose from the blood. Diabetics either do not produce enough insulin or their cells are insulin resistant, causing the blood glucose levels to rise. Perpetually high levels of blood glucose can lead to kidney damage, blindness, reduced blood circulation to the arms and legs, and nerve damage. The reduced blood circulation and nerve damage may cause foot problems.

Diabetics are more likely to have foot problems because their nerves are damaged and their feet may be numb to injury. Poor footwear or accidents may cause blisters or abrasions to their feet without a diabetic patient even being aware that the injury is occurring. Diabetic patients may not notice a foot injury until severe damage or infection develops.

Diabetes also reduces the body's ability to fight infections. Damage to blood vessels because of diabetes results in less blood and oxygen getting to the extremities. High blood sugar levels also prevent white blood cells, an important part of the immune system, from functioning normally. When these cells do not function properly, wounds take much longer to heal and become infected more frequently. Small sores or breaks in the skin may become deeper skin ulcers. The affected limb may even need to be amputated if these deep skin ulcers worsen.

Infections involving deep tissues and bone carry a higher risk of amputation. Ulcers larger than 1 inch across have a high risk of progressing on to limb amputation. Infections that involve gangrene generally must be amputated and also carry a high risk of death. Thus it is important to treat foot ulcers as early as possible before they become life threatening wounds.

Many larger hospitals have wound care centers specializing in the treatment of diabetic lower extremity wounds and ulcers. In these multidisciplinary centers, professionals of many specialties work with the patient in developing a treatment plan for the wound or leg ulcer. Treatment plans may include surgical debridement of the wound, improvement of circulation through surgery or therapy, special dressings, wound offloading, and antibiotics. Total care generally includes a combination of treatments.

Off-loading, in wound, care refers to relieving or removing pressure from an area of the body where there may be a wound or a bony protuberance causing pain. There are various options for offloading including; total contact cast (TCC), removable cast walker, Carville healing sandal, felted foam technique, and football dressing.

The Total Contact Cast (TCC) is a casting technique that is used to protect the foot and promote healing of diabetic foot ulcers. A plaster undercoat and fiberglass shell are applied in such a way as to contact the exact contour of the foot. Depending on the patient, a TCC is generally changed weekly or every two weeks until the ulcer is healed. TCCs are widely used because they have a high rate of successful healing. Although a TCC seems to be an effective off-loading modality, several disadvantages are known including: discomfort to the patient, occurrence of new ulcers, difficulty providing daily wound care, impairment of mobility, relatively high costs, and necessity of specialized staff. Prolonged casting may also cause joint rigidity and muscular atrophy. Furthermore, good blood circulation is required which may exclude the elderly, morbidly obese, and diabetic patients.

TCCs are not recommended in patients with deep abscess, gangrene, osteomyelitis, chronic venous stasis ulcers or severe peripheral arterial disease. Care should be taken when TCCs are used with elderly patients and patients who have an unsteady gait. The cumbersome rigidity of the TCC may cause these patients to unbalance and fall. Noncompliant patients who routinely miss scheduled appointments may also develop new ulcers that remain undetected under the TCC. The TCC also needs to be applied by an experienced cast technician or physician in order to avoid new skin breakdown and ensure patient safety, adding to the cost of the wound care.

There exists a need for an orthopedic boot that will provide the benefits of a TCC while incorporating the flexibility of a cam walker, a boot that expedites healing time yet is easy and comfortable to use.

### SUMMARY

An aspect of the invention generally relates to an orthopedic device and more specially a walking boot for off-loading a plantar ulceration. The walking boot may allow for near 100 percent off-loading of the affected area of the foot where the ulceration is located. The walking boot includes a rigid support shell and a unitary inner sole. The inner sole comprises individual cells. Each individual cell further comprises a rigid post and a compressible top layer. The rigid post attaches directly to the unitary inner sole of the rigid support shell.

The design of the inner sole allows for modification and configuration. The individual cells under an ulcer may be removed, which will off-load the plantar pressure under the ulcer(s). The removability of the individual cells of the inner sole accommodates for the size and shape of the ulceration, thereby reducing maximum peak pressure in the affected area during weight bearing.

Air bladders may be placed along the lateral, medial and posterior aspect of the heel of the sleeve to allow for padding and protection to the bony prominences of the ankle and heel. Another air bladder may be placed along the anterior aspect of the sleeve to allow padding and protection to the tibial crest.

Other aspects, advantages and novel features of the present disclosure will become apparent from the following detailed description of the disclosure when considered in conjunction with the accompanying drawings.

### BRIEF DESCRIPTION OF THE DRAWINGS

FIG. 1 is a top perspective view, taken from the user's left side, of an embodiment of the invention.

FIG. 2 is an exploded assembly view of an embodiment of the invention.

FIG. 3 is a bottom perspective view, from the user's left side, of the outer sole of an embodiment of the invention.

FIG. 4 is a front perspective view of an embodiment of the invention.

FIG. 5A is a right perspective view of an embodiment of the invention.

FIG. 5B is a left perspective view of an embodiment of the invention.

FIG. 6A is a perspective view of the cell array of an embodiment of the invention.

FIG. 6B is a magnified view of individual cell of the cell array of an embodiment of the invention.

FIG. 7 is a top view of the base of the shell of an embodiment of the invention.

FIG. 8A is a top perspective view from the user's left side of an embodiment of the invention showing an individual cell inserted into a seat on the shell.

FIG. 8B is a top perspective view from the user's left side of an embodiment of the invention showing the seated cell array with six cells removed where an ulceration would occur.

FIG. 9A is a left side view of an embodiment of the invention without the stabilizer attached.

FIGS. 9B-9D are left side views of embodiments of the invention with the stabilizer set at different desired positions.

FIG. 10 is a top perspective view from the user's left side of an embodiment of the invention showing the sleeve disengaged from the shell.

FIG. 11 is an isometric view of an embodiment of the invention that shows how the bottom portion of the sleeve connects to the shell.

FIG. 12A is a top perspective view of an embodiment of the invention with the straps and top portion of the sleeve unsecured.

FIG. 12B is a top perspective view of an embodiment of the invention showing the straps on the upper portion of the sleeve wrapped around in a clockwise direction around the outside of the stabilizers and securing to the back of the sleeve.

FIG. 13 is a left perspective view of one embodiment of a cell removal tool.

FIG. 14 is a side view of another embodiment of a cell removal tool.

### DETAILED DESCRIPTION OF THE EXEMPLARY EMBODIMENTS

Wounds tend to heal faster when the wound site is not subjected to shock and shear pressures. Wound off-loading of plantar ulcers is considered an essential step in promoting healing of the ulcer. The invention off-loads the ulcer by providing a configurable platform to support the injured foot. The platform comprises a cell array. The individual cells of the cell array have a cushioned top and a rigid stem to support the injured foot. Each cell is individually removable and replaceable, allowing treatment of ulcers at various places on the plantar area of the foot. Individually removable cells also allow a medical practitioner to accommodate for different size and shape ulcers.

Embodiments of the invention comprise a walking boot as a modality to assist in healing of foot ulcerations and musculoskeletal foot and ankle disorders. The walking boot accommodates the individual's foot and ankle condition, allowing mobility without compromising the healing process.

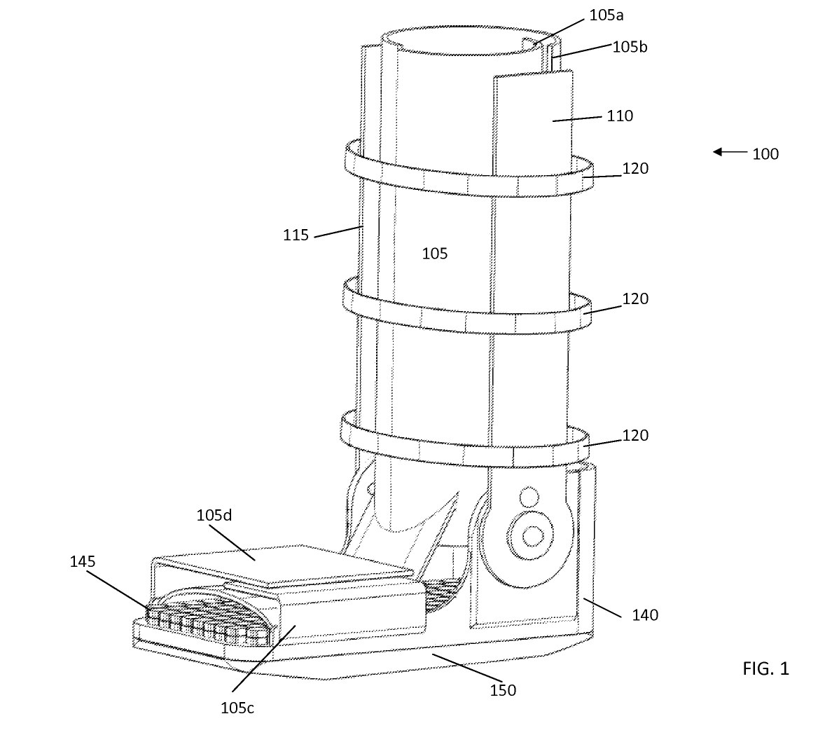

FIG. 1 illustrates a perspective view of the top left side of one embodiment of the invention. In FIG. 1, the walking boot 100 is shown fully assembled as it would look when placed on a patient. The walking boot 100 comprises three main parts a rigid support structure, a fabric sleeve, and a configurable inner sole.

The rigid support structure may be further broken down into shell 140 shaped like an open front mid-ankle boot and left and right stabilizers 110/115 attached to the ankle portion of the shell 140. Below the shell 140 is outer sole 150

The sleeve 105 has three main parts; an upper portion, a middle portion, and a lower portion. The upper portion may be further separated into left and right wings 105a and 105b and straps 120. The lower portion has left and right flaps 105c and 105d.

The inner sole is a cell array of individually removable cells 145 seated in holes formed in the base of the shell 140. Unlike the inner sole of a normal boot, the cell array is firmly seated in the holes and does not move from side to side or back and forth. The cells 145 provide a comfortable surface for the patient's foot and may be configured to off-load a plantar ulcer.

A patient's leg is placed in walking boot 100 with the patient's foot resting on cells 145 and the patient's lower leg wrapped inside sleeve 105. Straps 120 may be tightened around sleeve 105 to secure the lower leg in the walking boot 100. The lower flaps 105c/105d secures the patient's foot onto the cell array.

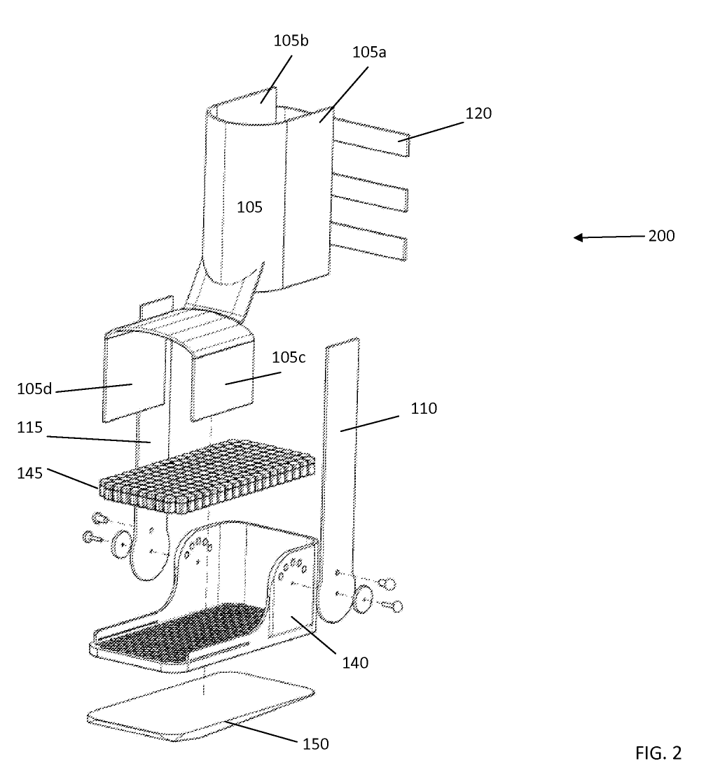

FIG. 2 is an exploded assembly view of the walking boot 200. The shell 140 is the centerpiece of the walking boot 200. The shell 140 is preferably made of a rigid but light material such as aluminum, stainless steel, titanium, black polycarbonate, graphite fiber, or glass fiber composites etc. Shell 140 has high tensile and impact strength. The other parts of the walking boot 200 are connected to the shell 140. Outer sole 150 is mounted to the bottom surface of the shell 140. The outer sole 150 may be affixed to the bottom of the shell 140 using a strong adhesive, for example rubber cement or silicon glue. Outer sole 150 may absorb some of the shock from walking and thus is preferentially made of some type of hard rubber or plastic.

Sleeve 105 is shown in FIG. 2 disengaged from the shell 140. Flaps 105a-105d are depicted in this exploded view in a disengaged state. Sleeve 105 may be made of a durable, flexible, stretchable fabric. Preferred fabrics include Gor-tex®, polyester, polypropylene, nylon, and neoprene. It may be advantageous to form sleeve 105 out of Gor-tex® because of the breathable and water resistant property of that fabric.

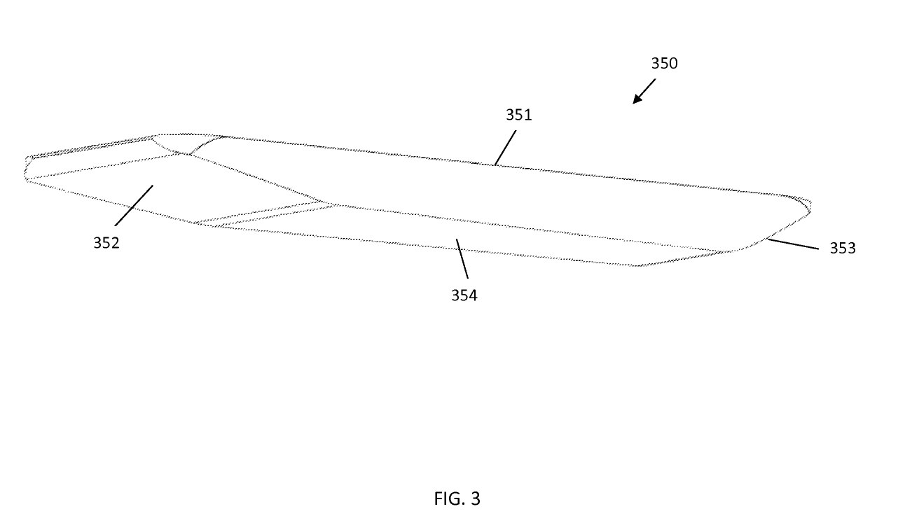

FIG. 3 is a left perspective view of outer sole 350. Outer sole 350 may be engineered of a resilient plastic or rubber material with a durometer of approximately 70 Shore A. Outer sole 350 may be designed with a substantially planar top surface 351, upwardly beveled front surface 352, upwardly beveled back surface 353, and substantially planar bottom surface 354. Beveled front and back surfaces 352,353 and bottom surface 354 form a rocker bottom. A rocker bottom is a configuration of the outer sole that will assist the user to propel forward when walking without having to plantarflex the forefoot on the ankle thus minimizing plantar grade pressure to the underlying ulceration.

Top surface 351 may be textured to more easily adhere to the bottom of shell 140. Alternatively, top surface 351 may be formed with patterns that are complementary to patterns on the bottom of shell 140 to form interlocking patterns, e.g. interlocking ridges. The interlocking patterns may help to bond the outer sole 350 to shell 140. Bottom surface 354 may be formed with raised patterns or treads to aid traction.

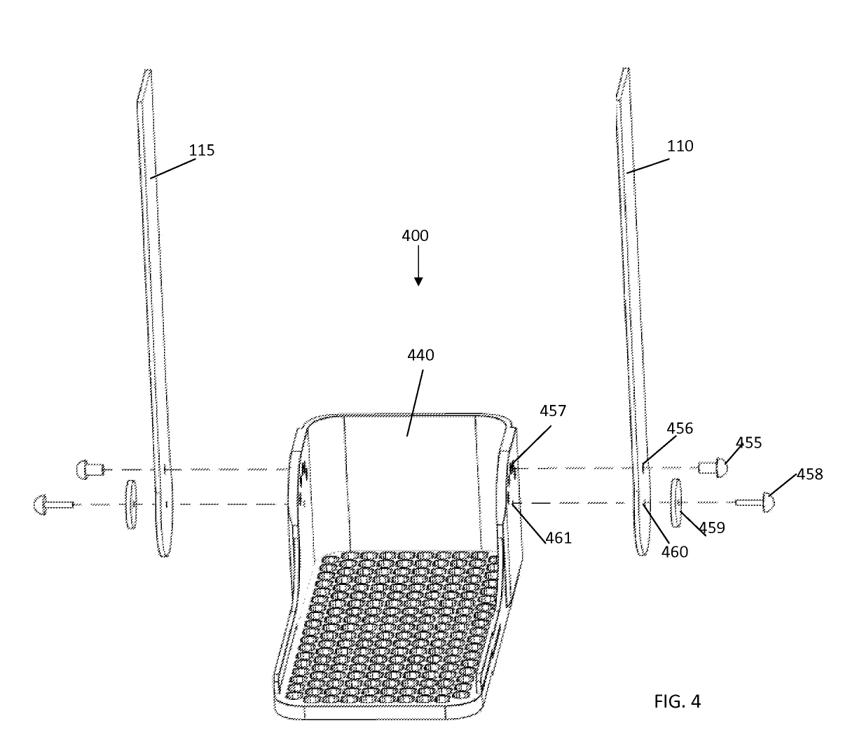

FIG. 4 is a front perspective view of walking boot 400. The two stabilizers 110,115 attach to the outside of shell 440, one on the left side of the ankle and one on the right side of the ankle. The stabilizers 110,115 are also preferably made of a rigid but light material such as aluminum, stainless steel, titanium, black polycarbonate, or graphite fiber or glass fiber composites. Left stabilizer 110 is attached to the shell 440 using permanent fastener 458 threaded through washer 459 and hole 460. The distal end of permanent fastener 458 anchors into hole 461. The permanent fastener 458 creates a pivot point about which left stabilizer 110 may rotate. In one embodiment, permanent fastener 458 may be a ⅛ inch rivet.

Temporary fastener 455 thread through hole 456 in left stabilizer 110 and fasten onto one of the holes 457 in shell 140. Holes 457 are placed in a semicircle on the side of shell 440. Holes 457, allows left stabilizer 110 to be set at different positions. The arrangement of permanent fastener 458, temporary fastener 455, holes 457 and 460 are mirrored on the opposite side for right stabilizer 115.

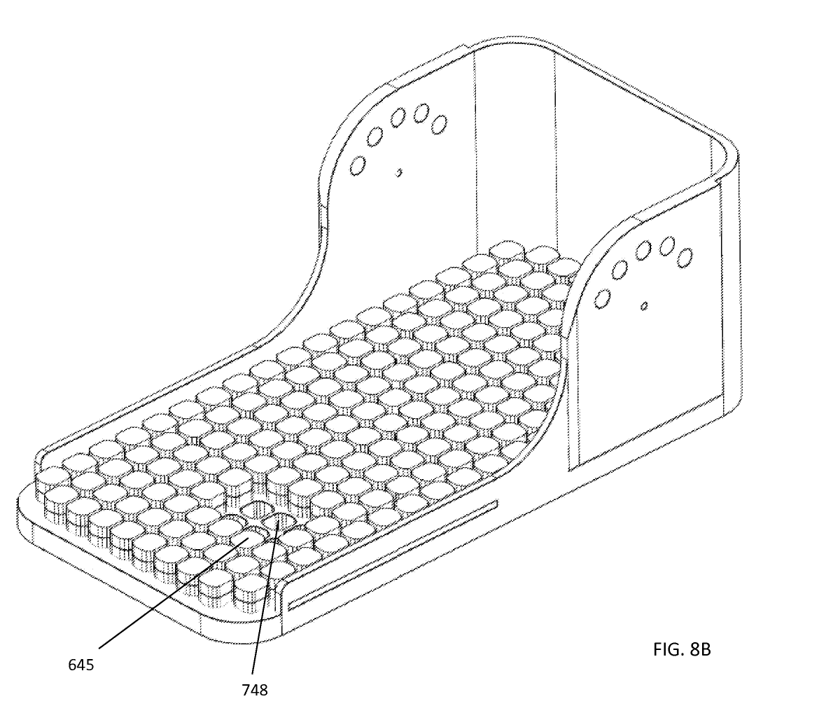

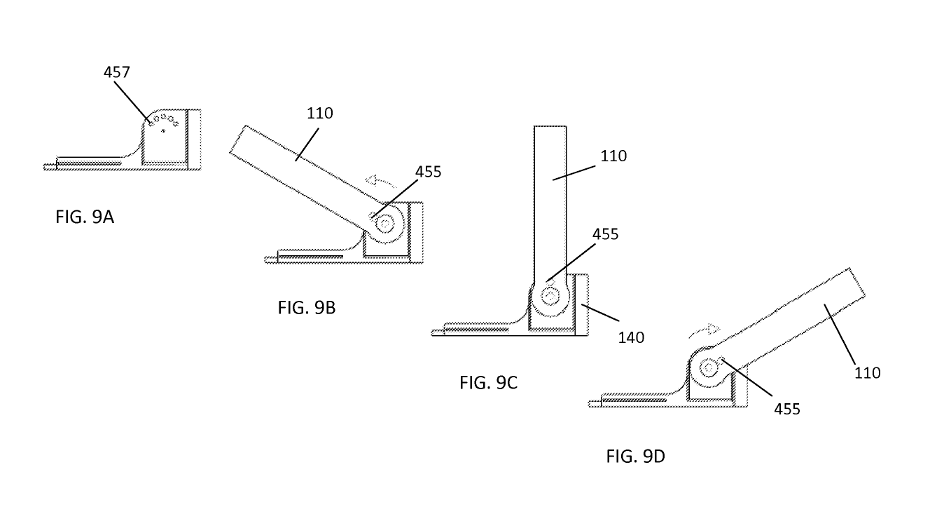

In one embodiment, temporary fastener 455 is a ¼ inch screw and holes 457 are ¼ inch holes. Stabilizer 110 may be rotated forward or backward and locked into place in a canted position by screwing temporary fastener 455 into the hole 457 corresponding to the canted position. More detail on changing the position of stabilizers 110,115 are given in the description of FIG. 9A-9D.

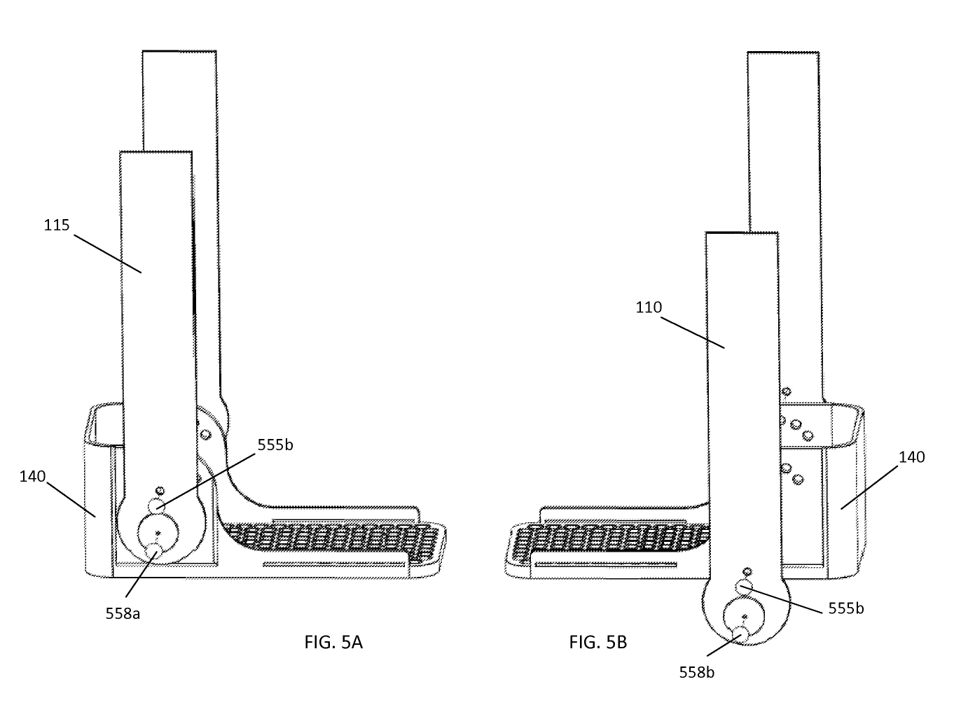

FIGS. 5A and 5B are right and left perspective views of the invention.

FIG. 5A shows the location where the right stabilizer 115 connects to the right side of the shell 140. FIG. 5B shows the location where the left stabilizer 110 connects to the left side of the shell 140. The stabilizers 110, 115 may be rotabably attached to shell 140 using permanent fasteners 558a and 558b.

In this illustration, the stabilizers are set in a fully upright position perpendicular to the base of shell 140. However, it should be known that the stabilizers may be set in other desired positions by loosening temporary fastener 555a, 555b, changing the position of the stabilizer 110,115, then tightening the temporary fastener at the new position.

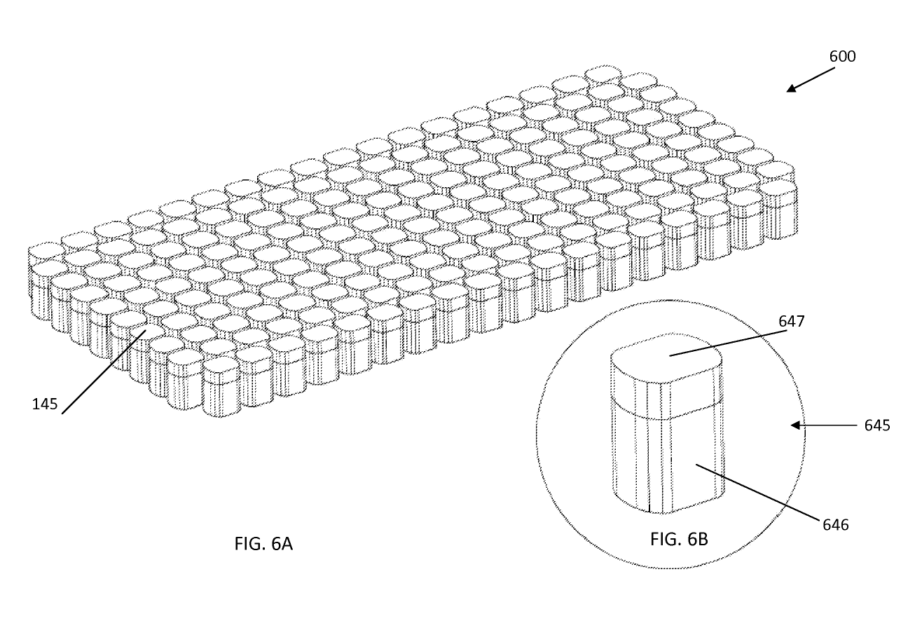

FIG. 6A is a perspective view of cell array 600. Cell array 600 comprises multiple individual cells 145 in tight formation. In one embodiment, the spacing between each cell 145 and the height of each cell 145 is uniform. It may be advantageous to uniformly space the cells 145 to provide uniform support for the patient's foot. Other embodiments may vary the spacing and height of the individual cells to accommodate different support needs. For example arch support may be included by increasing the height of some of the cells at the side of the array. In another embodiment the concentration of cells 145 may be increased in areas where greater plantar pressure is expected, e.g. underneath the ball of the feet and heel.

In FIG. 6B, a magnified view of the individual cell 645 is illustrated. In one embodiment the cell 645 may be comprised of a ½ inch thick post 646 of a rigid material such as black polycarbonate. Atop post 646 is a ¼ inch thick layer of a resilient cushion 647 such as Poron® (MSRF) material. The cushion 647 can be placed on the top of the post 646 with an adhesive. As explained above the height of the cell may be varied to accommodate the contours of the patient's foot. In such embodiments, the thickness of the post or the thickness of the cushion material may be changed depending on the area requiring the support.

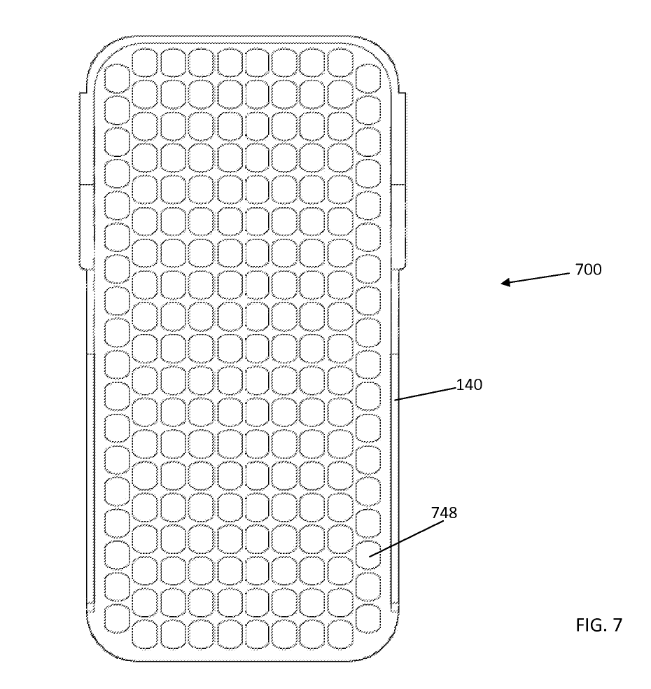

FIG. 7 is a top view of the inner sole 700 of an embodiment of the invention. Inner sole 700 is a unitary part of shell 140. In this illustration inner sole 700 is shown without the cell array attached. From this view, the individual recessed seats 748 are seen. Recessed seats 748 may be depressions cut or molded into the inner sole 700. In some embodiments, recessed seats 748 may be cutouts traversing the shell 140 from one side to the other, forming hollow tunnels through the shell 140. The size and shape of the recessed seats 748 substantially match the size and shape of the posts 646 of the individual cell 645. Recessed seats 748 are formed to be slightly larger than posts 646, yet sufficiently small to firmly hold the individual cells 645 once seated.

Individual cells 645 may be held in each recessed seat 748 in a variety of ways. In the exemplary embodiment, individual cells 645 are pushed into the recessed seat 748 and kept in place with an adhesive. In other embodiment, posts 646 may be friction fitted to the recessed seat 748 and may not require an adhesive to remain in place.

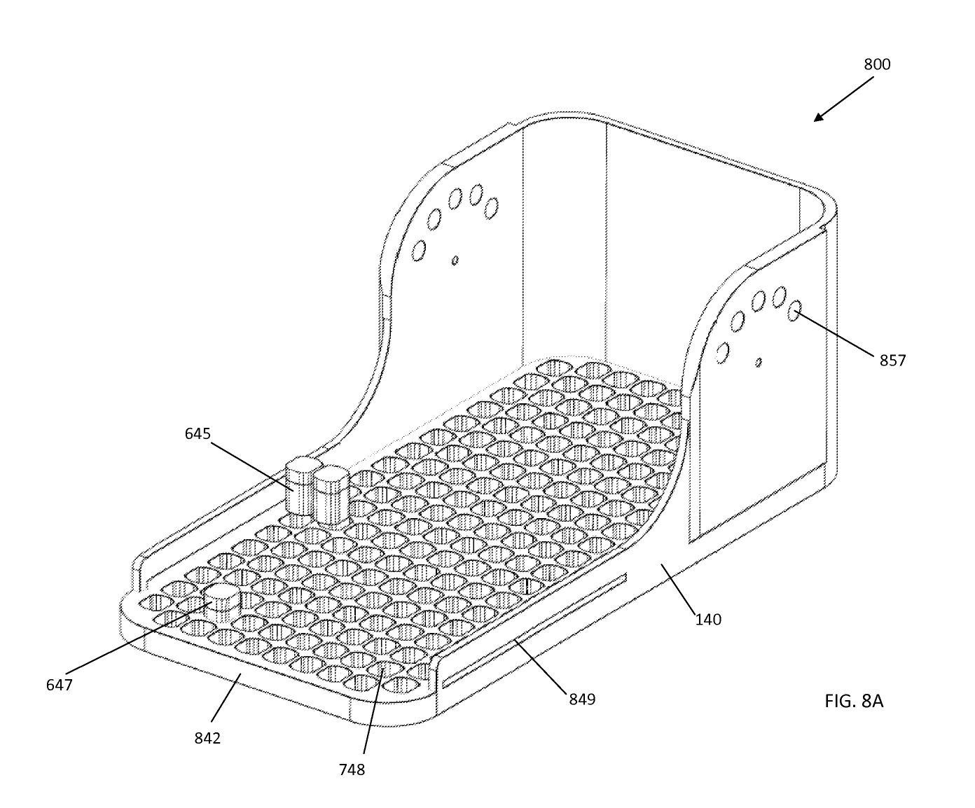

FIG. 8 is a top perspective view of another embodiment of the invention 800. Shell 140 is a unitary rigid structure physically resembling a boot with an open front. Shell 140 may be constructed of a rigid high tensile strength material. Ideally the material chosen should be light enough that older patients or patients who are otherwise incapacitated have some mobility. Some examples of shell 140 materials include; aluminum, stainless steel, titanium, black polycarbonate, graphite fiber, and glass fiber composites.

On the sides of the ankle portion of shell 140 are multiple position holes 857 positioned in a semicircle above pivot hole 461. Position holes 857 are adapted to receive temporary fasteners (not shown). Temporary fasteners may attach directly to position holes 857 or may attach to a backing placed behind position holes 857. Pivot hole 461 is adapted to receive a permanent fastener which roatably affixes stabilizers to the sides of shell 140.

The base 842 of shell 140 contains a plurality of recessed seats 748. Each recessed seat 748 is adapted to receive an individual cell 645. When firmly seated, the top of individual cell 645 protrudes from the recessed seat 748. As can be seen in FIG. 8, cushion 647 is exposed when individual cell 645 is seated in recessed seat 748. On each side of shell 140 are slits 849. Slits 849 completely traverse the side of shell 140 and in conjunction with the lower portion of the sleeve, secures the patients foot to the walking boot.

In FIG. 8B, the majority of the individual cells are seated in their recessed seats. Six of the recessed seats 748 are shown empty. The empty recessed seats may correspond to the size, shape, and location of a hypothetical plantar ulcer. The cells 645 around the empty seats 748 support the patient's foot while the empty seats 748 isolate the ulcer from shock and shear stress when the patient moves about. The ulceration should heal at a faster rate while protected in the pocket formed by the empty seats 748.

FIG. 9A is a left side views of shell 140 without stabilizer attached. In one embodiment of the invention, five position holes 457 are formed in the side of shell 140. Each hole 457 corresponds to a possible alignment position of stabilizer 110.

FIGS. 9B-9D are left side views of an embodiment of the invention showing left stabilizer 110 in various locked positions. These figures show various possible alignment positions of the left stabilizer 110. For example in FIG. 9B, temporary fastener 455 is fastened to the forward most position hole 457, canting left stabilizer 110 forward at an acute angle. In FIG. 9C, temporary fastener 455 is fastened to the middle position hole 457, resulting in stabilizer 110 standing in an upright position perpendicular to the base of shell 140. In FIG. 9D, temporary fastener 955 is fastened to rearmost position hole 457 canting the left stabilizer 110 backward at an acute angle. Temporary fastener 455 working in conjunction with position holes 457 provide a method of locking the stabilizer 110 at various desired positions. Stabilizers may be locked in a canted position when treating a medical condition that would require the user's foot/ankle to be in a plantarflexed or dorsiflexed position e.g. Post-Achilles tendon rupture repair where the desired position of the ankle is plantarflexed to decrease tension across the Achilles repair allowing optimization for healing.

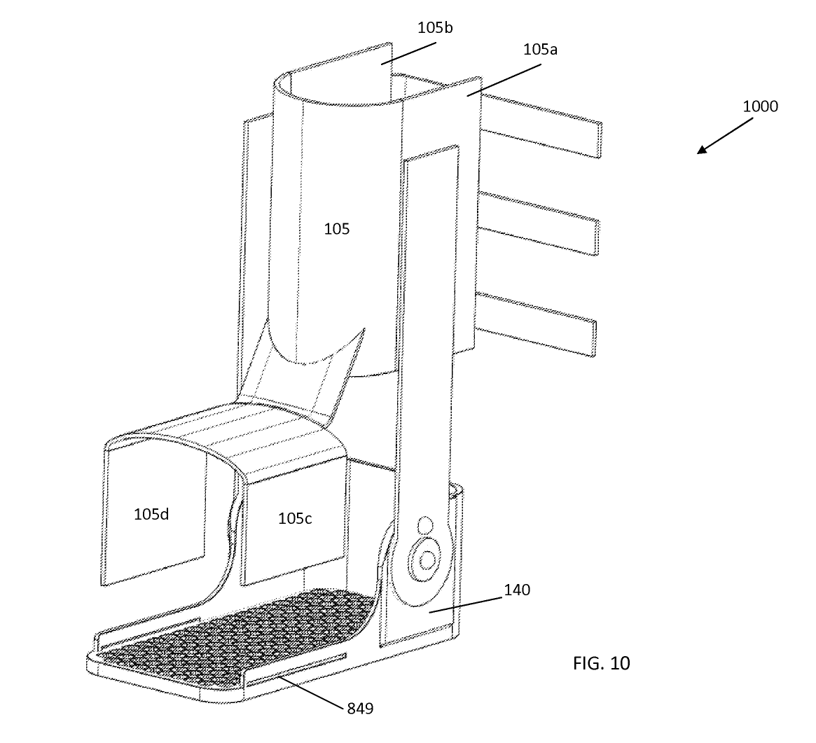

FIG. 10 is a top perspective view of the left side of one embodiment of the invention 1000, depicting sleeve 105 disengaged from shell 140. Sleeve 105 may be made of many types of fabrics, but is preferably elastic, breathable, durable, and light weight. The material may be made more sheer or perforated to increase breathability. Preferred fabrics include Gortex®, polyester, polypropylene, nylon, and neoprene. Sleeve 105 comprises three major portions; upper, middle and lower.

In one embodiment of the invention, the upper portion of the sleeve 105 resembles a U, with the bottom of the U facing forward. The left wing 105a and right wing 105b of the upper portion are preferably of sufficient length to wrap around the calf of a patient. The upper portion further comprises straps 120. Straps 120 are attached at one end to the upper portion of sleeve 105 and are detached or loose at the distal end. Straps 120 may be wrapped around the left and right stabilizers 110/115 securing the patients limb to the walking boot 1000. Securing means may include buckles, snaps, or hook and loop a.k.a. Velcro® attached to the straps 120. Preferably securing means is adjustable, reusable, and releasable.

The middle portion of sleeve 105 connects the upper and lower portions. The middle portion may be a flat strip of fabric attached at one end to the upper portion and the other end to the lower portion of sleeve 105.

The lower portion of sleeve 105 also resembles a U with the bottom of the U facing upward. The left and right lower flaps 105c and 105d respectively, are designed to fit into slots on 849 located on both sides of the shell 140. In the depicted embodiment, the lower flaps 105c, 105d are asymmetrical with the right flap 105d longer than the left flap 105c.

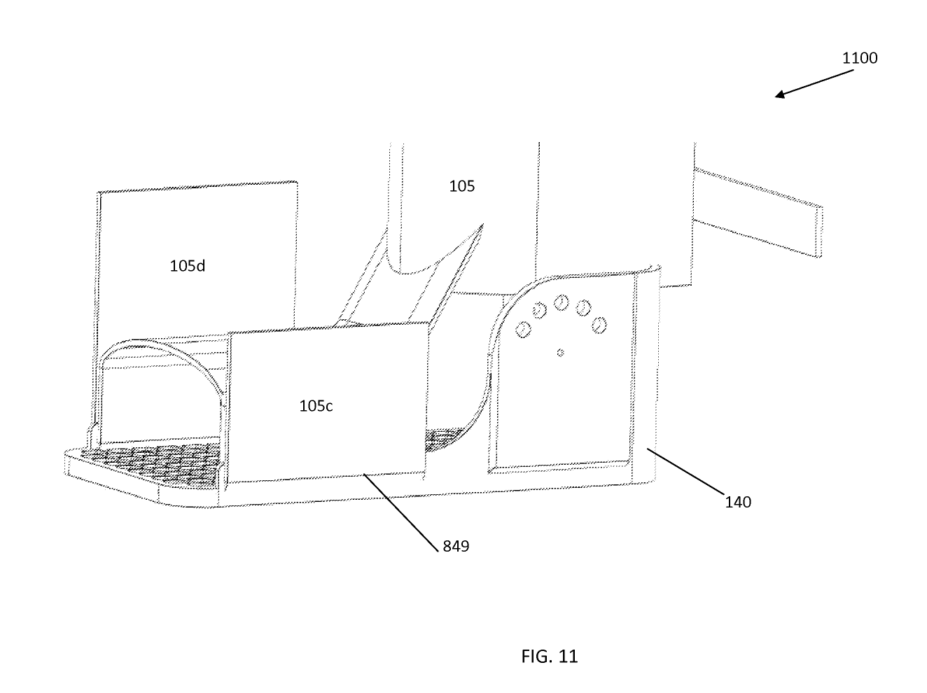

FIG. 11 is an isometric view of the invention 1100. Right and left lower flaps 105c,105d of the sleeve 105 are inserted and pulled through the slits 849. An adjustable, reusable, and releasable securing means such as buckles, snaps, or hook and loop fasteners may be attached to the ends of the flaps 105c,105d. The flaps 105c,105d may be inserted through slits 849 from inboard to outboard, i.e. from inside shell 140 towards the outside of shell 140.

In one method of securing a patient's foot to the invention 1100, the patient's foot is placed into the shell 140, on top of the cell array. Lower flaps 105c, 105d are inserted through the slits 849 from inboard to outboard. The lower flaps are then pulled vertically, tightening the lower portion of sleeve 105 over the patient's foot. Lower flaps 105c,105d may then be secured by a reusable securing means. For example, if hook and loop fasteners are used, left flap 105c would secure to the top of the sleeve 105 and right flap 105d would secure to the top of flap 105c, as seen in FIG. 1.

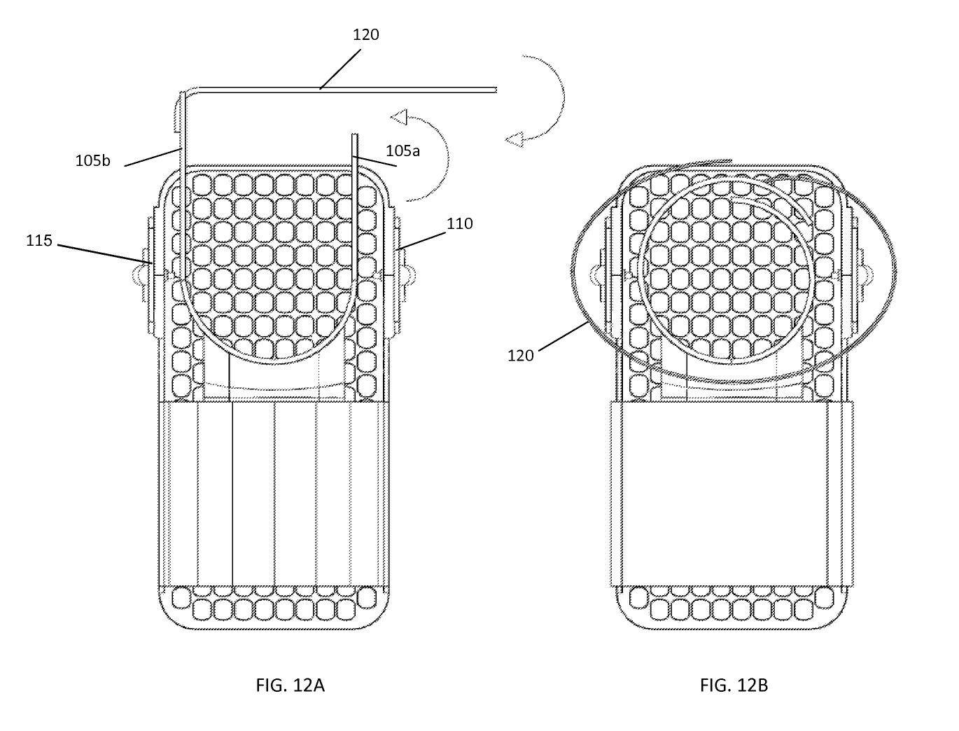

FIGS. 12A and 12B are a top views depicting how the upper portion of the sleeve 105 is fastened to stabilizers 110, 115. In FIG. 12A the upper portion of the sleeve 105 is shown in an open position ready for insertion of a patient's leg. The arrows show how the right and left wings 105a,105b of the upper portion of the sleeve 105 are secured around the patient's leg. The right wing 105b may be designed slightly longer than the left wing 105a. The straps 120 are directly connected at one end to the right wing 105b.

In FIG. 12B, the sleeve 105 and straps 120 are depicted as they would look if wrapped around a patient's leg below the knee. When wrapped around the patient's leg, the slightly longer right wing 105b overlaps the left wing 105a. The straps 120 are designed to wrap around the outside of the stabilizers 110,115 in a clockwise direction. Depending on the securing means used, the straps 120 may wrap around stabilizers 110,115 then fasten to the back of the sleeve 105 or fasten back onto themselves. For example, if using hook and loop fasteners, a set of hooks may be attached to the inward face of strap 120 near the tip of the free edge. Complimentary loops may be attached to the outward face of sleeve 105 or the outward face of strap 120 so that the strap fastens to themselves.

### Method of Operation

Offloading, in its most basic form is preventing pressure on a wound. When the wound is a plantar ulcer, the foot is generally immobilized with a cast to prevent the ulcer from contacting the ground while walking. Generally debriding the hyperkeratotic skin surrounding the wound edge is the one of the first procedure of offloading. When left untreated, the thick and callused edges of a wound may roll inward and inhibit the wound edges from migrating towards the center. This hyperkeratotic edge may also cause increased plantar pressure.

The center of a wound may seem to be the area in most need of offloading, but many plantar ulcers have such significant depth that the weight bearing forces do not affect the center of the wound. Due to the natural offloading of the central wound cavity, plantar foot pressures intensify at the leading edge of the wound. Frequent debridement of the hyperkeratotic edge may be required to relieve pressure from the wound edges.

Referring back to FIG. 1, a method of applying one embodiment of the invention to a plantar ulcer is presented. A physician or other trained professional examines the patient to determine the size, depth, location, severity, etc of the plantar ulcer. Debridement of the skin surrounding the ulcer is performed if needed.

The cell array is modified to accommodate the ulcer. One or more cells 145 may be removed from the cell array such that when the patient's foot is secured in the walking boot, the ulcer does not contact the cells 145. It may be advantageous to remove sufficient cells so that the edge of the ulcer does not contact the cell array.

Once the cell array has been modified to fit the ulcer, the walking boot 100 is ready to be secured to the patient. The sleeve is loosened from the walking boot 100 by undoing the straps 120, left and right upper wings 105a/105b, and left and right lower flaps 105c/105d. The physician places the patient's foot into the shell of the boot, on top of the modified cell array.

If the stabilizers 110,115 need adjustment, the physician loosens the temporary fasteners 455 and rotates the stabilizers 110,115 to the desired orientation. The temporary fasteners 455 are then tightened to fix the stabilizers 110,115 in the new position.

Once the stabilizers 110 are properly positioned, the sleeve 105 is tightened around the patient's leg. The physician wraps the upper portion of the sleeve 105 around the patient's leg below the knee. The left wing 105a is wrapped around the leg first followed by the right wing 105b, so that a portion of the right wing 105b overlaps the left wing 105a. Straps 120 are wrapped clockwise around the left stabilizer 110 and around to the right stabilizer 115. The straps 120 are secured to the sleeve 105 or alternatively back unto themselves.

Once the upper sleeve is secured, the patient's foot should be secured using the lower portion of the sleeve 105. One method of securing the patient's foot to the cell array 145 involves taking the U shaped portion of the lower portion of the sleeve 105 and laying it over the foot. Left flap 105c and right flap 105d may then be inserted though their respective slits 849 of the shell 140 going from inboard to outboard. Left flap 105c is then pulled up around the shell 140 and secured onto the top surface of the U shaped lower portion of the sleeve 105. Right flap 105d may then be pulled up around the shell and secured to the top of the left flap 105c. Pulling up on left and right flaps 105c/105d tightens the U shaped portion of the sleeve over the patient's foot.

### Removing and Replacing Cells

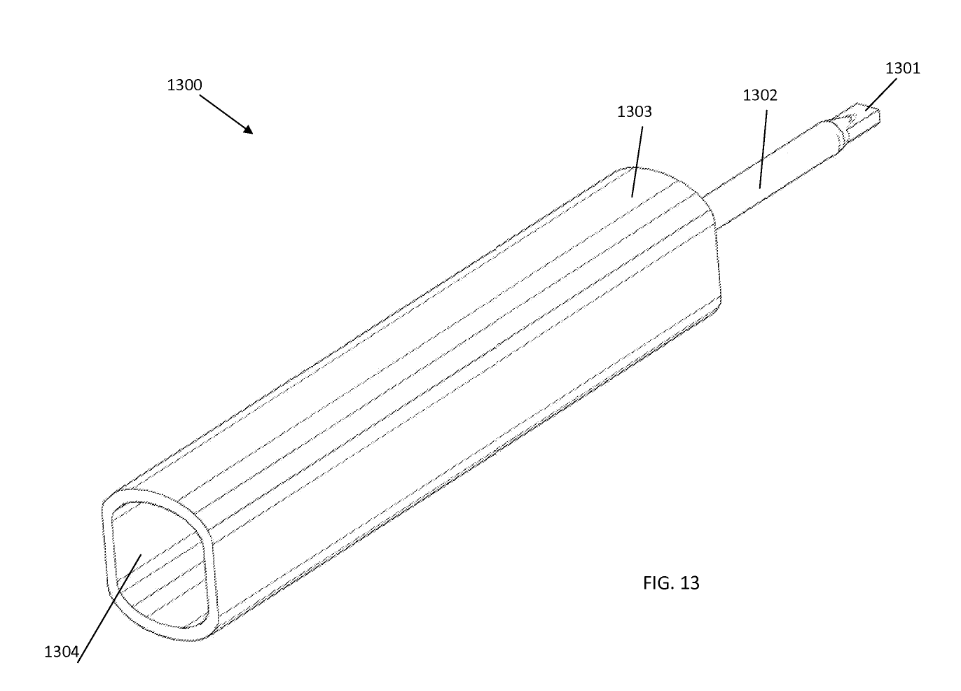

In another embodiment of the invention, the walking boot 100 includes a tool 1300 for removing and replacing the cells 145 of the cell array 600. The tool 1300 may be a dual purpose tool. Referring to FIG. 13, tool 1300 is illustrated in a perspective view. Tool 1300 includes a tool tip 1301, a shaft 1302, and a cylinder 1303 with an opening 1304 at one end of the cylinder 1303.

Tool tip 1301 is designed to fit the temporary fasteners 455 of FIG. 4. In this embodiment, tool tip 1301 is depicted as a flat-head screwdriver. Depending on the type of temporary fastener used, tool tip 1301 may be another shape such as a Phillips screwdriver, hex, star, or square. To use the tool tip 1301, the end of the tool tip 1301 is inserted into the compatibly shaped temporary fastener 455 and rotated. Preferably, a clockwise rotation tightens the temporary fastener 455 and a counter-clockwise rotation loosens the temporary fastener 455.

Shaft 1302 extends the reach of tool tip 1301 and also connects the tool tip 1301 to the cylinder 1303. Shaft 1302 is preferably molded of a high tensile strength material to withstand torque from tightening and loosening the temporary fastener 455.

Attached to shaft 1302 is cylinder 1303. Cylinder 1303 is a hollow cylinder sized and shaped to match the size and shape of the individual cells 145,645 (FIGS. 1 and 6) of the cell array. Cylinder 1303 may be gripped and used as a handle when using the tool tip 1301.

To remove individual cells 145,645, opening 1304 is placed over the resilient cushion portion 647 of one of the individual cell 145, 645. Opening 1304 is sized to tightly fit the individual cell 145,645 such that friction holds the individual cell 145,645 in the hollow opening 1304 of the cylinder 1303. Lifting up on tool 1300 will pull the individual cell 145,645 from its recessed seat 748. Individual cell 145, 645 may be removed from the tool by firmly grasping post 646 and pulling the individual cell 145, 645 out of cylinder 1303.

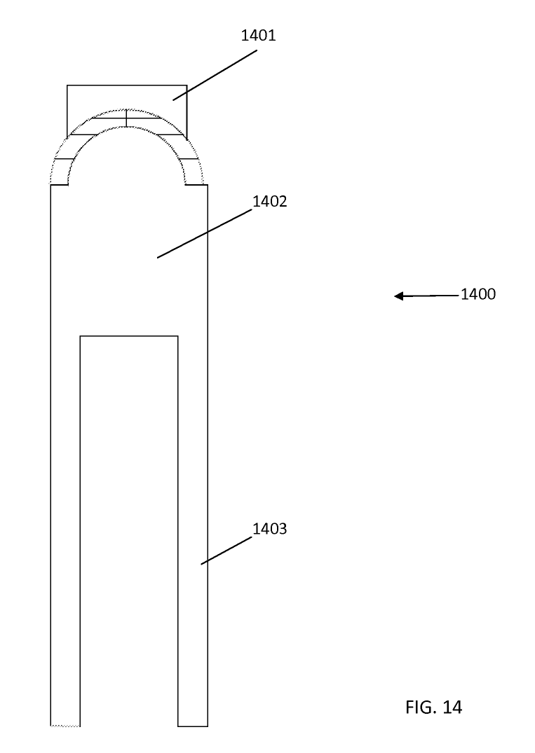

Referring now to FIG. 14 a top view of an alternate embodiment of a cell removal tool 1400 is shown. Cell removal tool 1400 operates much like tweezers. At one end of tool 1400 is a tool tip 1401 depicted as a flat-head driver. As with tooltip 1301, tooltip 1401 may be another shape such as a Phillips screwdriver, hex, star, or square. Below tooltip 1401 is the tool body 1402. Tool body 1402 may be many different shapes, for example cylindrical or rectangular. Tool body 1402 may double as a handle, thus is it may be advantageous to form tool body as an ergonomic shape for easier operation. Attached to tool body 1402 is a pair of opposing pincers 1403. The pincers 1403 form the operational end of cell removal tool 1400. Force applied to the pincers 1403 cause them to bend inward. In operation opposing pincers 1403 may be slid between individual cells 145,645. The pincers 1403 are then squeezed so that they firmly grasp the sides of the individual cells 145,645 and the tool 1400 is pulled upward, extracting the individual cell 145,645.

### CONCLUSION

While this specification includes many specifics, these should not be construed as limitations on the scope of the disclosure or of what may be claimed, but rather as descriptions of features specific to particular implementations of the disclosure. Certain features that are described in this specification in the context of separate implementations may also be implemented in combination in a single implementation. Conversely, various features that are described in the context of a single implementation may also be implemented in multiple implementations, separately or in sub-combination. Moreover, although features may be described above as acting in certain combinations and even initially claimed as such, one or more features from a claimed combination may in some cases be excised from the combination, and the claimed combination may be directed to a sub-combination or variations of a sub-combination. Accordingly, the claimed invention is limited only by the claims that follow below.

What is claimed is:

1. An orthopedic walking boot for off-loading plantar ulcers of diabetic patients, comprising:
a rigid shell further comprising a boot portion and two adjustable stabilizers attached to and extending from the ankle region of the boot;
a sleeve with an upper portion and a lower portion, the upper portion further comprising one or more straps to secure the sleeve to the stabilizers, the lower portion adapted to couple to two slits in the sides of the boot;
a cell array comprising individually removeable and replaceable cells, each cell further comprising a resilient top portion and a rigid support post to be received by a recessed seat formed in the boot portion.

2. The orthopedic walking boot of claim 1, wherein the rigid support frame is made of one of the following materials; stainless steel, titanium, black polycarbonate, graphite fiber or glass fiber.

3. The orthopedic walking boot of claim 1, wherein the two adjustable stabilizers are rotatably attached to the rigid shell at a pivot point by a permanent fastener.

4. The orthopedic walking boot of claim 1, wherein the position of the two adjustable stabilizers are set by temporary fasteners.

5. The orthopedic walking boot of claim 4, wherein the temporary fasteners are screws that are loosened to release the stabilizers and tightened to lock the stabilizer in a desired position.

6. The orthopedic walking boot of claim 1, wherein the lower portion of the sleeve covers the top of the patient's foot and further comprises two flaps that inserts into slits in the shell.

7. The orthopedic walking boot of claim 1, wherein the resilient top portion of each individual cell is made of a microcellular urethane and the rigid support post is polycarbonate.

8. The orthopedic walking boot of claim 5, wherein the recessed seats are indentations or cutouts in the base of the boot adapted to fit and firmly hold the rigid support posts.

9. A method of off-loading plantar ulcers of diabetic patients using a configurable orthopedic walking boot comprising:
determining the size and shape of the plantar ulcer;
removing one or more cells of a cell array corresponding to the size and shape of the plantar ulcer, wherein the cells of the cell array are seated in recessed seats formed in the configurable walking boot;
adjusting a pair of stabilizers to a desired position and locking the stabilizers into that position;
placing a patients leg into a sleeve of the configurable orthopedic walking boot and securing the patient's foot onto the cell array;

10. A method of claim 9, wherein adjusting a pair of stabilizers further comprises, loosening a temporary fastener on each stabilizer, adjusting the desired position of stabilizers, and tightening temporary fasteners at the new position.

11. The method of claim 9, wherein the patients leg is placed between two flaps of an upper portion of the sleeve, the flaps are folded around the patients leg, and straps attached to the upper portion of the sleeve are wrapped around the stabilizers, leg, and sleeve.

12. The method of claim 9, wherein the patient's foot is secured to the cell array by placing a lower portion of the sleeve over the patient's foot, inserting two flaps of the lower portion of the sleeve into two slits in the sides of the boot, and pulling up on the flaps to tighten the lower portion over the top of the patient's foot.

13. The method of claim 12, wherein the two flaps of the lower portion are inserted through the two slits inboard to outboard.

14. The method of claim 9, wherein the recessed seats are indentation or cutouts formed in the walking boot.

15. The method of claim 9, wherein removing one or more cells is performed with a cell replacement tool provided with the walking boot.

16. An orthopedic walking boot for off-loading plantar ulcers of diabetic patients, comprising:
a rigid shell further comprising a boot portion and two adjustable stabilizers attached to and extending from the ankle region of the boot;
the boot portion further comprising an integral sole of uniform recesses coupled to rigid support posts of a cell array;
the cell array further comprising individually removeable and replaceable cells, each cell further comprising a resilient top portion and the rigid support post; and
a soft flexible cover comprising two portions, an upper sleeve portion adapted to wrap around the lower calf of a patient and a lower cover portion adapted to cover the top of the patients foot;
the upper sleeve portion further comprises one or more straps to secure the patients calf to the adjustable stabilizers and the lower cover portion comprises two wings to fit through two slits in the rigid shell and secure the patients foot to the rigid shell.

17. The orthopedic walking boot of claim 16, wherein the rigid support frame is made of one of the following materials; stainless steel, titanium, black polycarbonate, graphite fiber or glass fiber.

18. The orthopedic walking boot of claim 16, wherein the two adjustable stabilizers are rotatably attached to the shell at a pivot point by a permanent fastener.

19. The orthopedic walking boot of claim 16, wherein the position of the two adjustable stabilizers are set by temporary fasteners.

20. The orthopedic walking boot of claim 19, wherein the temporary fasteners are screws that are loosened to release the stabilizers and tightened to lock the stabilizer in a desired position.
